(19) United States
(12) Patent Application Publication
(10) Pub. No.: US 2022/0051795 A1
(43) Pub. Date: Feb. 17, 2022

(54) ANALYSIS SYSTEM AND ANALYSIS METHOD

(71) Applicant: Hitachi, Ltd., Tokyo (JP)

(72) Inventors: Syuntaro YUI, Tokyo (JP); Takanobu OOSAKI, Tokyo (JP); Hideyuki BAN, Tokyo (JP)

(21) Appl. No.: 17/274,796

(22) PCT Filed: Apr. 9, 2019
(86) PCT No.: PCT/JP2019/015504

§ 371 Date: Mar. 10, 2021

(30) Foreign Application Priority Data
Sep. 12, 2018 (JP) 2018-170590

(51) Int. Cl. G16H 50/20; G16H 10/60; G06F 9/30

(52) U.S. Cl. CPC G16H 50/20; G06F 9/3001; G16H 10/60

(57) ABSTRACT

An analysis system that analyzes an effect of a diagnosis and treatment action and measure is implemented by a computer including an arithmetic device configured to execute predetermined processing and a storage device connected to the arithmetic device. The analysis system includes: an input unit configured to receive an analysis condition; an event detection unit configured to extract a disease developing event; and a cost calculation unit configured to calculate a cost of a diagnosis and treatment action related to a disease name to be analyzed that is generated after a period of the disease developing event extracted by the event detection unit.

### CROSS-REFERENCE TO RELATED APPLICATIONS

The present application claims priority from Japanese Patent Application No. 2018-170590 filed on Sep. 12, 2018, contents of which are incorporated into the present application by reference.

### TECHNICAL FIELD

The present invention relates to an analysis system that analyzes effects of diagnosis and treatment actions and measures in a medical field.

### BACKGROUND ART

Due to a rapid change in a population structure such as declining birthrate and aging population, it is an urgent task to construct a sustainable medical care providing system in response to a rapid increase in medical costs. Given the variety of stakeholders, it is necessary to design a system based on evidence to formulate a new system that guarantees the quality of medical care and optimizes medical costs. It is a global activity to use stored data, and data analysis is considered as an effective method for generating evidence. A high-quality medical care providing system can be constructed by providing incentives and penalties for effective diagnosis and treatment actions or measures having a high effect and extracted by data analysis. In a current effectiveness analysis, the analysis is executed depending on whether a diagnosis and treatment action is executed or depending on a providing amount of the diagnosis and treatment action.

The following related arts are described as a background art in the present technical field. PTL 1 (JP-A-2014-215761) discloses a method for analyzing a situational change of a market. According to the method, time series of situation vectors including numeric values each for characterizing a rate of increase or decrease in market occupancy scale of each competition element at each time point are calculated, a time series model of the situation vectors sequentially from a competition element in an uppermost level toward lower levels is created by referring to a hierarchical order relation in market competition of service category hierarchical information storage units when detecting a change in situation vectors for a given time section, and a change point in the situation vectors is detected as a situational change.

PTL 2 (WO2015/77582) discloses a composition and a method for treating and improving symptoms of rheumatoid arthritis by using an antibody that specifically binds to a human interleukin-6 receptor (hIL-6R).

NPL 1 discloses a technique for extracting risk factors of diabetes using Naive Bayes and binary logistic regression.

### CITATION LIST

### Patent Literature

PTL 1: JP-A-2014-215761
PTL 2: WO2015/77582
NPL 1: B. Lee et al., “Identification of Type 2 Diabetes Risk Factors Using Phenotypes Consisting of Anthropometry and Triglycerides based on Machine Learning”, IEEE J. of Biomedical and Health Informatics, vol. 20. No. 1 Jan. 2016

### SUMMARY OF INVENTION

### Technical Problem

It is required to accurately evaluate an economic value of a measure or diagnosis and treatment action considered to be highly effective when determining an incentive method for the diagnosis and treatment action. However, since a database indicated by a medical bill stores data including much data loss, called real world data, it is difficult to calculate an accurate economic evaluation by simply adding an amount of money described in the medical bill. For example, a disease developing period cannot be accurately known due to data loss, and various complications occurring after the disease develops cannot be known.

Although NPL 1 discloses a method for extracting risk factors of diabetes based on examination values, the presence or absence of a diagnosis and treatment action or measure, and time series information of the diagnosis and treatment action or measure are not considered. Although PTL 1 discloses a system that detects a change point of a KPI and extracts a causal relationship, a temporal change of the causal relationship is not considered. Although an economic evaluation specialized for a component called Sarilumab is calculated in PTL 2, an economic evaluation is not considered for a medicine whose component is not specialized, or a diagnosis and treatment action. As described above, from the viewpoint of an overall economic loss due to disease developing, it is difficult to effectively and efficiently present an economic evaluation of a highly effective measure or diagnosis and treatment action.

Therefore, the invention provides a technique for effectively and efficiently presenting economic value of the highly effective measure or diagnosis and treatment action by using a medical care database including data loss.

### Solution to Problem

A representative example of the invention disclosed in the present application is as follows. That is, an analysis system that analyzes an effect of a diagnosis and treatment action and measure is implemented by a computer including an arithmetic device configured to execute predetermined processing and a storage device connected to the arithmetic device. The analysis system includes an input unit configured to receive an analysis condition; an event detection unit configured to extract a disease developing event; and a cost calculation unit configured to calculate costs of a diagnosis and treatment action related to a disease name to be analyzed that are generated after a period of the disease developing event extracted by the event detection unit.

### Advantageous Effect

According to an aspect of the invention, an economic effect for each diagnosis and treatment action can be presented, and materials that contribute to planning of efficiency improvement of medical costs can be presented. Problems, configurations, and effects other than those described above will become apparent from the following description of embodiments.

### BRIEF DESCRIPTION OF DRAWINGS

FIG. 1 is a configuration diagram showing an economic value evaluation system for a diagnosis and treatment action and a measure according to an embodiment of the invention.

FIG. 2 is a hardware configuration diagram showing the economic value evaluation system for the diagnosis and treatment action and measure according to the present embodiment.

FIG. 3 is a flowchart schematically showing processing executed by the economic value evaluation system for the diagnosis and treatment action and measure according to the present embodiment.

FIG. 4 is a sequence diagram showing a part of the processing (S302 to S305) shown in FIG. 3.

FIG. 5 is a diagram showing an example of a condition setting and processing result display screen.

FIG. 6 is a flowchart showing step S302 in detail.

FIG. 7 is a diagram showing an example of diagnosis and treatment action data.

FIG. 8 is a diagram showing an example of clinical data.

FIG. 9 is a diagram showing an example of disease developing knowledge data.

FIG. 10 is a diagram showing an example of a disease developing date management table.

FIG. 11 is a flowchart showing step S303 in detail.

FIG. 12 is a sequence diagram showing a part of processing (S3031 and S3032) shown in FIG. 11.

FIG. 13 is a diagram showing processing of generating a diagnosis and treatment action time series information table.

FIG. 14 is a diagram showing an example of a diagnosis and treatment action feature amount table.

FIG. 15 is a flowchart showing step S306 in detail.

FIG. 16 is a flowchart showing step S307 in detail.

FIG. 17 is a diagram showing an example of a related disease name table.

FIG. 18 is a diagram showing an example of a medical cost table.

FIG. 19 is a flowchart showing step S308 in detail.

FIG. 20 is a diagram showing an example of a condition setting and processing result display screen.

FIG. 21 is a diagram showing an example of a condition setting and processing result display screen.

FIG. 22 is a flowchart showing step S308 in detail according to a first modification.

FIG. 23 is a diagram showing an example of a condition setting and processing result display screen according to the first modification.

FIG. 24 is a diagram showing another example of the condition setting and processing result display screen according to the first modification.

FIG. 25 is a flowchart showing step S308 in detail according to the first modification.

FIG. 26 is a diagram showing an example of a condition setting and processing result display screen according to a second modification.

FIG. 27 is a diagram showing an example of a condition setting and processing result display screen according to the second modification.

### DESCRIPTION OF EMBODIMENTS

### First Embodiment

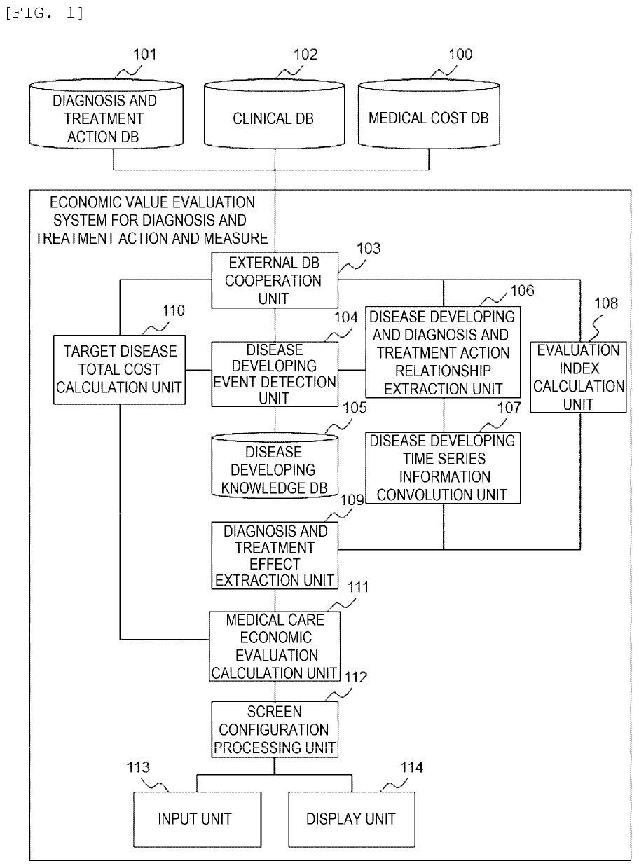

FIG. 1 is a configuration diagram showing an economic value evaluation system for a diagnosis and treatment action and a measure according to an embodiment of the invention.

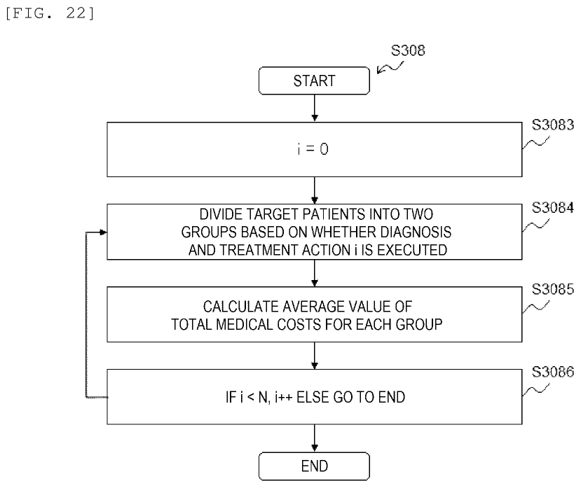

The system includes an external DB cooperation unit 103, a disease developing event detection unit 104, a disease developing knowledge database 105, a disease developing and diagnosis and treatment action relationship extraction unit 106, a disease developing time series information convolution unit 107, an evaluation index calculation unit 108, a diagnosis and treatment effect extraction unit 109, a target disease total cost calculation unit 110, a medical care economic evaluation calculation unit 111, a screen configuration processing unit 112, an input unit 113, and a display unit 114. The external DB cooperation unit 103 has a function of cooperating with a database outside the system. For example, the external DB cooperation unit 103 acquires data stored in a diagnosis and treatment action database 101, a clinical database 102, and a medical cost database 100, and may cooperate with other databases. In the economic value evaluation system for the diagnosis and treatment action and measure according to the present embodiment, the disease developing and diagnosis and treatment action relationship extraction unit 106, the disease developing time series information convolution unit 107, the evaluation index calculation unit 108, and the diagnosis and treatment effect extraction unit 109 are not essential configurations, but are configurations necessary to display a selected index (an importance degree) of a highly effective diagnosis and treatment action or a measure on condition setting and processing result display screens (FIGS. 20, 23, 24, and 26).

The input unit 113 is an interface that receives an input from a user. The display unit 114 is an interface that outputs an execution result of a program in a format that can be visually recognized by the user.

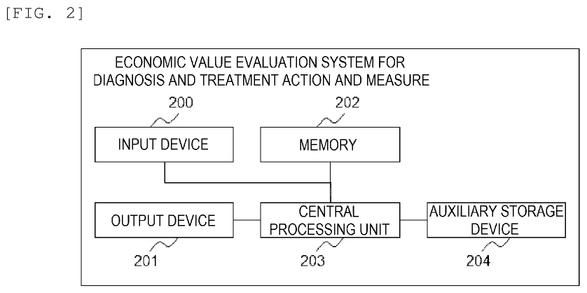

A hardware configuration of the system will be described. FIG. 2 is a hardware configuration diagram showing the economic value evaluation system for the diagnosis and treatment action and measure according to the present embodiment.

An input device 200 is a keyboard, a mouse, a pen tablet, or the like that forms the input unit 113, and is an interface that receives an input from the user. An output device 201 is a display device such as a liquid crystal display device or a cathode-ray tube (CRT) that forms the display unit 114, and is an interface that outputs an execution result of a program in a format that can be visually recognized by the user. Alternatively, the output device 201 may be a device that outputs the execution result onto a paper medium such as a printer. The input device 200 and the output device 201 may be provided in a terminal connected to the economic value evaluation system for the diagnosis and treatment action and measure via a network.

A central processing unit 203 is a processor (an arithmetic device) configured to execute a program. Specifically, the processor executes programs to implement the external DB cooperation unit 103, the disease developing event detection unit 104, the disease developing and diagnosis and treatment action relationship extraction unit 106, the disease developing time series information convolution unit 107, the evaluation index calculation unit 108, the diagnosis and treatment effect extraction unit 109, the target disease total cost calculation unit 110, the medical care economic evaluation calculation unit 111, and the screen configuration processing unit 112. A part of processing performed by the processor executing the programs may be performed by another type (for example, by hardware) of an arithmetic device (such as a field programmable gate array (FPGA) and an application specific integrated circuit (ASIC)).

A memory 202 includes a ROM which is a non-volatile storage element and a RAM which is a volatile storage element. The ROM stores an invariable program (for example, a BIOS), or the like. The RAM is a high-speed and volatile storage element such as a dynamic random access memory (DRAM), and temporarily stores a program to be executed by a processor 11 and data to be used when the program is executed.

An auxiliary storage device 204 is a large-capacity and non-volatile storage device such as a magnetic storage device (for example, an HDD) and a flash memory (for example, an SSD). The auxiliary storage device 204 stores data to be used when the central processing unit 203 executes a program and the program to be executed by the central processing unit 203. Specifically, the auxiliary storage device 204 stores the disease developing knowledge database 105. A part of or the entire of the disease developing knowledge database 105 is stored in the memory 202 in a short time as the program is executed. In addition, the program is read from the auxiliary storage device 204, loaded into a memory, and executed by the central processing unit 203.

Although not shown, the economic value evaluation system for the diagnosis and treatment action and measure includes a communication interface that controls communication between the economic value evaluation system for the diagnosis and treatment action and measure and other devices in accordance with a predetermined protocol.

The program to be executed by the central processing unit 203 is introduced into the economic value evaluation system for the diagnosis and treatment action and measure via a removable medium (such as a CD-ROM and flash memory) or a network, and is stored in the non-volatile auxiliary storage device 204 which is a non-transitory storage medium. Therefore, the economic value evaluation system for the diagnosis and treatment action and measure may include an interface that reads data from the removable medium.

The economic value evaluation system for the diagnosis and treatment action and measure is a computer system formed by one physical computer or a plurality of logical or physical computers, or may be operated on a virtual computer constructed on a plurality of physical computer resources.

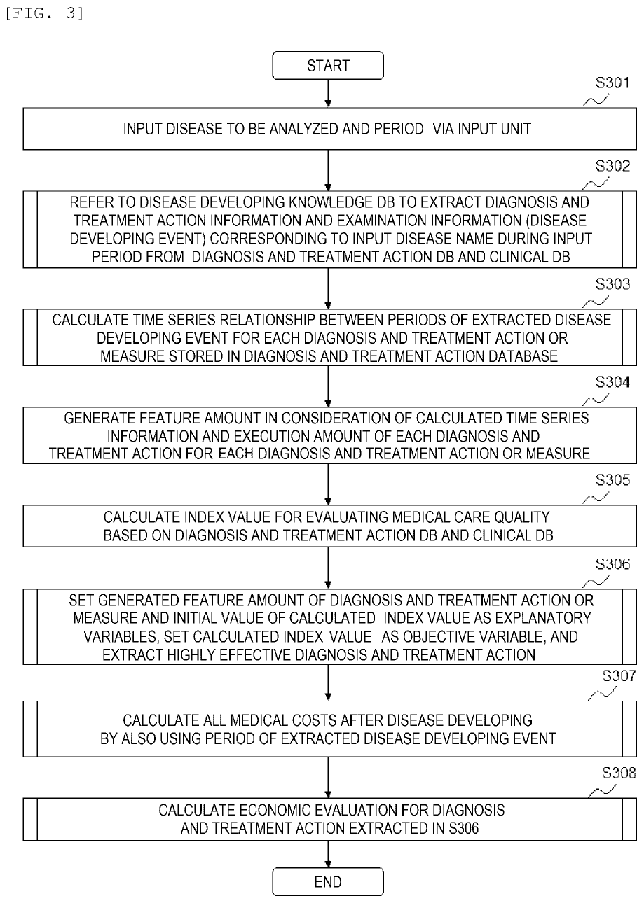

FIG. 3 is a flowchart schematically showing processing executed by the system according to the present embodiment. FIG. 4 is a sequence diagram showing a part of the processing (S302 to S305) shown in FIG. 3.

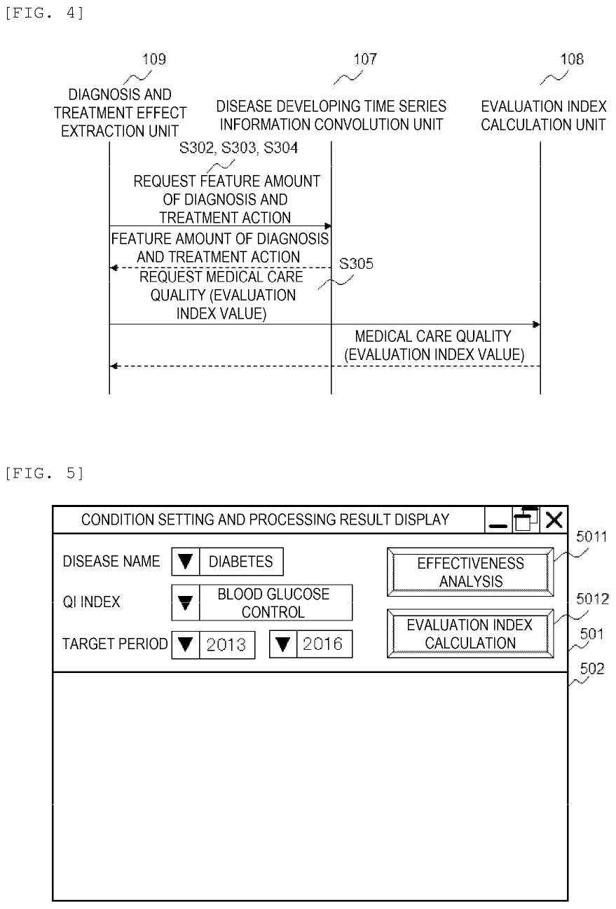

First, when the display unit 114 displays a condition setting and processing result display screen (FIG. 5), the user inputs a disease (a disease name) to be analyzed, a QI index, and a period via the input unit 113 (S301).

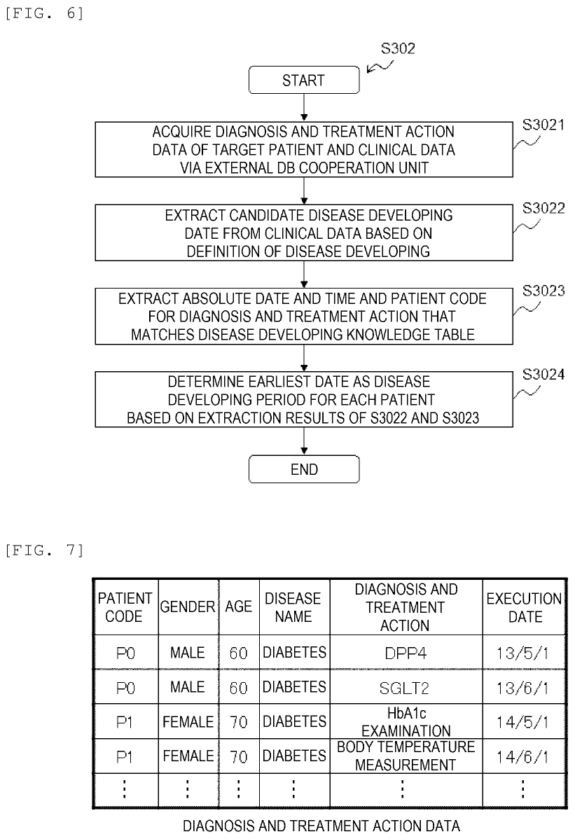

Next, the disease developing event detection unit 104 refers to the disease developing knowledge database 105 to extract diagnosis and treatment action information and examination information corresponding to the input disease name during the period to be analyzed input in step S301 from the diagnosis and treatment action database 101 and the clinical database 102 (S302). That is, information (a disease developing event) of a patient who may have developed the disease (the disease name) is extracted in step S302. The processing in step S302 will be described in detail with reference to FIG. 6.

Then, the disease developing and diagnosis and treatment action relationship extraction unit 106 calculates a time series relationship (for example, relative date and time indicating a temporal relationship) with periods of the disease developing event extracted in S302 for each diagnosis and treatment action or measure stored in the diagnosis and treatment action database 101 (S303). The processing in step S303 will be described in detail in FIG. 11.

Next, the disease developing time series information convolution unit 107 generates a feature amount in consideration of the time series information calculated in S303 and an execution amount of each diagnosis and treatment action or measure for the diagnosis and treatment action or measure processed in S303 (S304).

Then, the evaluation index calculation unit 108 calculates an index value for evaluating medical care quality based on the diagnosis and treatment action database 101 and the clinical database 102 (S305).

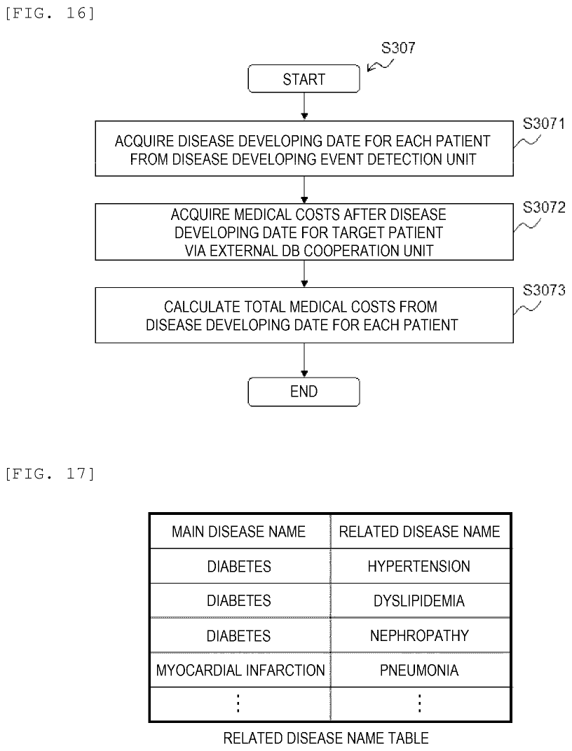

Next, the diagnosis and treatment effect extraction unit 109 sets the feature amount of the diagnosis and treatment action or measure generated in S304 and an initial value of a feature amount of the index value calculated in S305 as explanatory variables, sets an effect feature amount (for example, the index value) calculated in S305 as an objective variable, and extracts a highly effective diagnosis and treatment action or measure (S306). The processing in step S306 will be described in detail with reference to FIG. 15. The initial value of the feature amount of the index value is used to recognize a change amount of the index value during the period to be analyzed, and can fill a difference between basic values among patients. If there is no difference between the basic values among patients, it is not necessary to use the initial value.

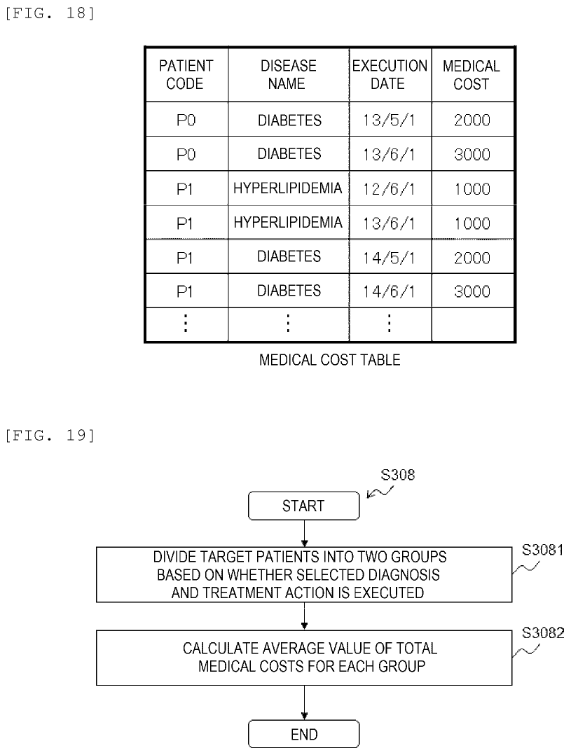

Then, the target disease total cost calculation unit 110 calculates, using the period of the extracted disease developing event, all medical costs after the disease develops (S307). The processing in step S307 will be described in detail with reference to FIG. 16.

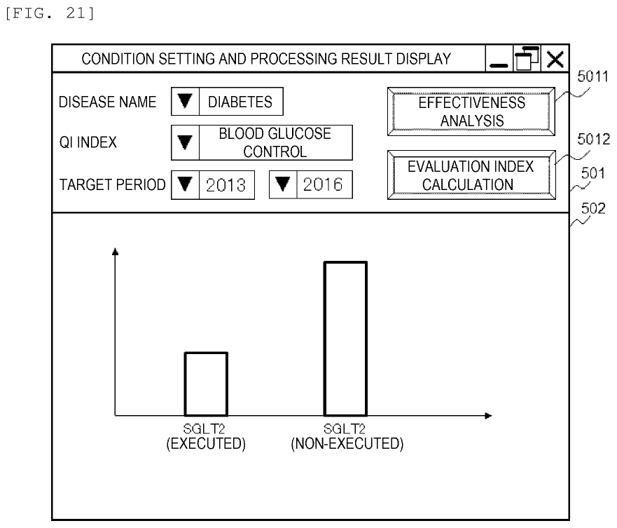

Finally, the medical care economic evaluation calculation unit 111 calculates an economic evaluation for the diagnosis and treatment action extracted in S306 (S308). The processing in step S308 will be described in detail in FIG. 19.

FIG. 5 is a diagram showing an example of the condition setting and processing result display screen displayed by the display unit 114 in step S301.

The condition setting and processing result display screen includes a condition setting region 501 and a processing result presentation region 502. The condition setting region 501 displays an “effectiveness analysis” button 5011 that is operated to analyze effectiveness of a diagnosis and treatment action, an “evaluation index calculation” button 5012 that is operated to evaluate an economic value of a diagnosis and treatment action or measure, and pull-down selection input fields for setting an analysis condition. In the example shown in FIG. 5, a condition is set for extracting a diagnosis and treatment action or measure having a high effect of affecting blood glucose control by using data of a diabetes patient from year 2013 to year 2016, and nothing is displayed in the processing result presentation region 502.

Next, step S302 will be described in detail with reference to FIG. 6.

First, the disease developing event detection unit 104 acquires diagnosis and treatment action or measure data of a target patient from the diagnosis and treatment action database 101 and acquires clinical data from the clinical database 102 via the external DB cooperation unit 103 (S3021).

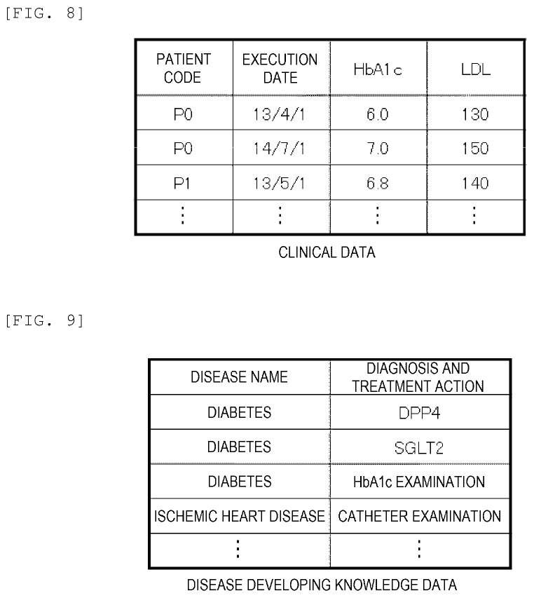

As shown in FIG. 7, the diagnosis and treatment action data stored in the diagnosis and treatment action database 101 includes patient attributes (such as a patient code, gender, and age), a diagnosis and treatment action (such as a prescribed medicine and an examination content), and an execution date. In addition to the diagnosis and treatment action executed by a medical care institution, the diagnosis and treatment action data may include measures other than the diagnosis and treatment action (for example, health guidance, diet guidance, and regular exercise). As shown in FIG. 8, the clinical data stored in the clinical database 102 includes a patient code, an examination execution date, and an examination value. The diagnosis and treatment action database 101 is not limited to one in which all diagnosis and treatment actions or measures performed on a patient are correctly input, and may be one including data loss. An examination recorded in the clinical database 102 may not be regularly undergone by a patient. That is, an input loss may occur in either one of the diagnosis and treatment action database 101 and the clinical database 102.

Next, the disease developing event detection unit 104 extracts a candidate disease developing date from the clinical data based on a definition of the disease developing (S3022). In the case of diabetes, a threshold of a value of HbAlc is 6.5% or more as defined in clinical guidelines. July, 2014 is extracted as a candidate disease developing date for a patient having a patient code P0, and May, 2013 is extracted as a candidate disease developing date for a patient having a patient code P1.

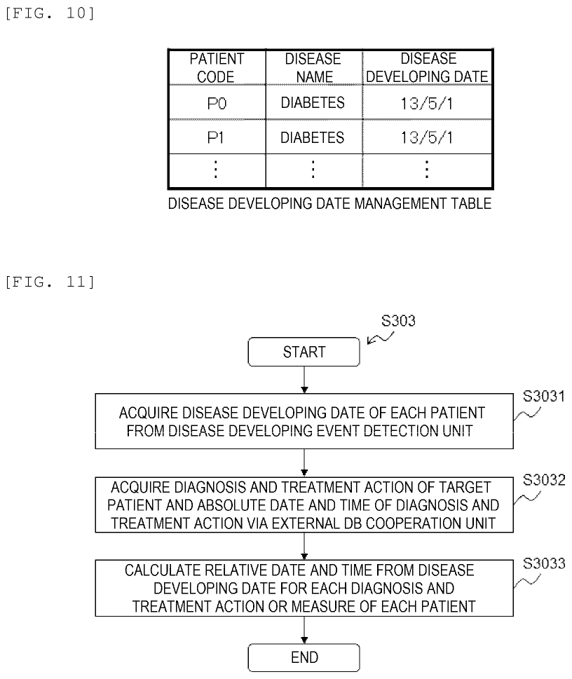

Then, the disease developing event detection unit 104 extracts absolute dates (an execution date) and a patient code for a diagnosis and treatment action or measure that matches disease developing knowledge data extracted from the disease developing knowledge database 105 (S3023). As shown in FIG. 9, the disease developing knowledge data records a relationship between a disease and a diagnosis and treatment action or measure. Specifically, a DPP 4 inhibitor prescription, an SGLT2 inhibitor prescription, and an HbAlc examination are recorded as diagnosis and treatment actions related to diabetes. (P0, DPP4, 13/5/1), (P0, SGLT2, 13/6/1), (P1, HbAlc examination, 14/5/1) can be extracted by using data shown in FIGS. 7 to 9. Since body temperature measurement is not defined in the disease developing knowledge data, (P1, body temperature measurement, 14/6/1) is not extracted in step S3023.

Although the disease developing knowledge database 105 is registered in advance in the present embodiment, the diagnosis and treatment action or measure related to a disease may be extracted using the diagnosis and treatment action data and the clinical data. For example, a disease developing knowledge generation unit may be provided. The disease developing knowledge generation unit may refer to the diagnosis and treatment action data and the clinical data to extract a diagnosis and treatment action or measure related to a disease and construct the disease developing knowledge database 105. Specifically, a diagnosis and treatment action or measure having a high correlation with a disease name in the diagnosis and treatment action data is extracted, or a diagnosis and treatment action or measure having a high correlation with a disease developing definition (for example, a value of the HbAlc is 6.5% or more) in the clinical data is extracted.

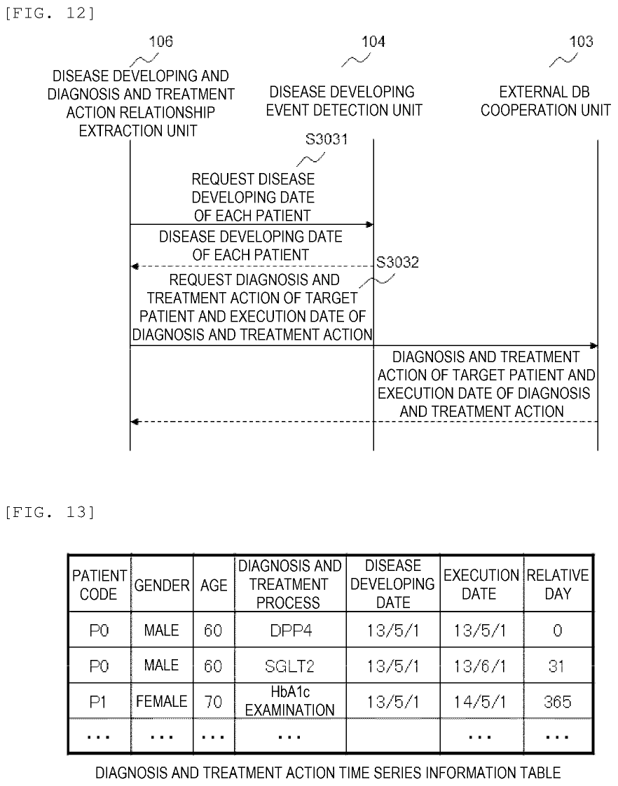

Next, an earliest date and time is determined as a disease developing period for each patient based on the candidate disease developing date extracted in S3022 and the absolute date extracted in S3023 (S3024). In this example, a disease developing date of the patient having the patient code P0 is Jul. 1, 2014 when a value of HbAlc is 6.5% or more according to the definition in the guidelines. However, since a DPP4 inhibitor prescription date extracted in S3023 is May 1, 2013, May 1, 2013 is recorded in a disease developing date management table (FIG. 10) as a disease developing date.

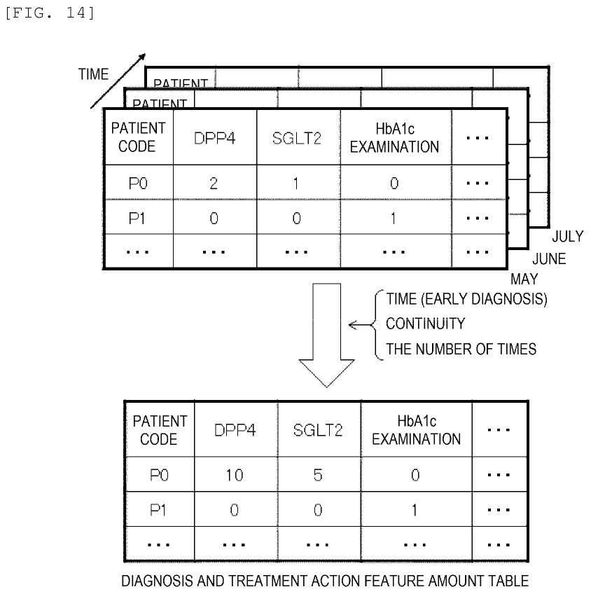

Next, step S303 will be described in detail. FIG. 11 is a flowchart showing step S303 in detail. FIG. 12 is a sequence diagram showing a part of processing (S3031 to S3032) shown in FIG. 11.

First, the disease developing and diagnosis and treatment action relationship extraction unit 106 acquires a disease developing date (for example, the disease developing date management table shown in FIG. 10) of each patient from the disease developing event detection unit 104 (S3031).

Next, the disease developing and diagnosis and treatment action relationship extraction unit 106 accesses the clinical database 102 via the external DB cooperation unit 103, and acquires a diagnosis and treatment action or measure of a target patient and absolute dates of the diagnosis and treatment action or measure (S3032). In the example shown in FIG. 7, (P0, DPP4, 13/5/1), (P0, SGLT2, 13/6/1), (P1, HbAlc examination, 14/5/1), (P1, body temperature measurement, 14/5/1) are acquired.

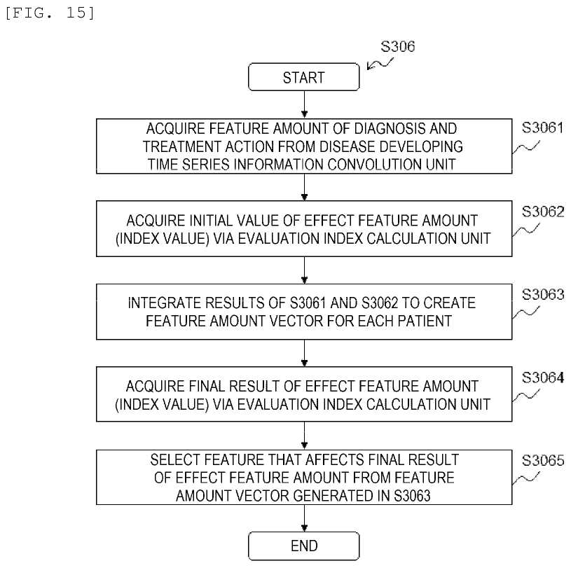

Finally, the disease developing and diagnosis and treatment action relationship extraction unit 106 calculates relative days from a disease developing date to an execution date of each diagnosis and treatment action or measure for each patient (S3033). The calculated relative days are recorded in a diagnosis and treatment action time series information table shown in FIG. 13.

Next, two methods to be used in step S304 will be described in detail. A first method focuses on an importance of an early diagnosis and treatment, and a second method focuses on an importance of continuously executing a diagnosis and treatment action or measure.

The first method focuses on the importance of the early diagnosis and treatment. When the disease developing time series information convolution unit 107 generates a data set of explanatory variables, the disease developing time series information convolution unit 107 weights and adds instances of diagnosis and treatment actions or measures executed in an early stage even when the diagnosis and treatment actions are the same. Accordingly, an early diagnosis and an early treatment are emphasized and the data set of explanatory variables obtained by compressing time series components is generated. Specifically, a feature amount Xij is calculated for each diagnosis and treatment action or measure j by using the following Formula 1. According to Formula 1, the shorter the relative days from the disease developing date to the execution date, the larger the feature amount Xij of the diagnosis and treatment action or measure. The diagnosis and treatment action or measure that contributes to an early diagnosis and an early treatment can be weighted heavily.

$$Xij = {\sum }_{t} f ( t ) Rij ( t ) \\ Rij ( t ) = \{ \begin{matrix}1 & if Aj was executed at the related month \\ & t ( = Execution\_Month ( Aj ) - M ( i ) ) \\ 0 & otherwise\end{matrix} \\ f ( t ) = ⌊ t divide A ⌋ , ( A is constant . ) \qquad [ Formula 1 ]$$

When M(i) is set as a diabetes developing date of a patient i and a diagnosis and treatment action or measure group A is set as {A1, A2 . . . AJ} (for example, A1=DPP4, A2=SGLT2), a relative day Rij(t) from the diabetes developing date can be calculated, and the feature amount Xij can be calculated by multiplying the relative day Rij (t) with a monotone decreasing function f(t) serving as a weighting element.

The second method focuses on a continuously executed diagnosis and treatment action or measure. When the disease developing time series information convolution unit 107 generates a data set of explanatory variables, the disease developing time series information convolution unit 107 weights and adds instances of continuously executed diagnosis and treatment actions or measures even when the diagnosis and treatment actions are the same. Accordingly, continuity of the diagnosis and treatment actions is emphasized and the data set of explanatory variables obtained by compressing time series components is generated. Specifically, a feature amount Xij is calculated for each diagnosis and treatment action or measure j by using the following Formula 2. According to Formula 2, the larger a feature amount Xij of a diagnosis and treatment action or measure executed multiple times at a regular interval, the smaller a feature amount Xij of the diagnosis and treatment action or measure executed multiple times at irregular intervals or executed intensively during one period, and a diagnosis and treatment action or measure whose continuity contributes to improvement of a medical condition can be weighted heavily. In Formula 2, Rij(t) is defined in the same manner as the Rij (t) in Formula 1, and is weighted using a monotone decreasing function f(t) that decreases with elapse of time as an element.

$$Xij = \frac{{( {\sum }_{t} f ( t ) Rij ( t ) )}^{2}}{{\sum }_{t} {( f ( t ) Rij ( t ) )}^{2}} \\ Rij ( t ) = \{ \begin{matrix}1 & if Aj was executed at the related month \\ & t ( = Execution\_Month ( Aj ) - M ( i ) ) \\ 0 & otherwise\end{matrix} \\ f ( t ) = ⌊ t divide A ⌋ , ( A is constant . ) \qquad [ Formula 2 ]$$

The feature amount calculated in step S304 is recorded in a diagnosis and treatment action feature amount table shown in FIG. 14. As shown in FIG. 14, the feature amount calculated in Formula 2 is generated by compressing time series components in consideration of temporal closeness (an early diagnosis), continuity, and the number of times of diagnosis and treatment actions or measures organized in time series.

Next, step S305 will be described in detail. The evaluation index calculated in step S305 is an index for evaluating medical care quality, and is referred to as a quality indicator or the like. For example, a percentage of diabetes patients whose blood glucose control of HbAlc is less than 6.5% is used in the field of diabetes. Therefore, the evaluation index calculation unit 108 acquires clinical data from the clinical database 102 via the external DB cooperation unit 103. The evaluation index calculation unit 108 calculates an evaluation index to be 1 if a value of the HbAlc is 6.5% or more and calculates an evaluation index to be 0 if a value of the HbAlc is less than 6.5%.

Next, step S306 will be described in detail with reference to FIG. 15.

First, the diagnosis and treatment effect extraction unit 109 acquires the feature amount of a diagnosis and treatment action or measure (a diagnosis and treatment action feature amount table shown in FIG. 14) generated by the disease developing time series information convolution unit 107 (S3061).

Next, an initial value of an effect feature amount is acquired via the evaluation index calculation unit 108 (S3062). In the example shown in FIG. 5, since a target period is specified from year 2013 on the condition setting and processing result display screen (FIG. 5), an evaluation index of year 2013 is acquired. As described above, an initial value of a feature amount of an index value may be used freely.

Then, a result of S3061 and a result of S3062 are integrated to create a feature amount vector for each patient (S3063). The feature amount vector to be used as an explanatory variable is generated in this manner, so that an existing selection method can be used, and the implementation to the system becomes easy.

Next, a final result of the effect feature amount is acquired via the evaluation index calculation unit 108 (S3064). In the example shown in FIG. 5, since the target period is specified until year 2016 on the condition setting and processing result display screen (FIG. 5), an evaluation index of year 2016 is acquired.

Finally, a feature that affects a final result of the effect feature amount is selected from the feature amount vector generated in S3063 (S3065). Specifically, a feature amount vector output in S3063 is selected as an explanatory variable and a variable output in S3064 is selected as an objective variable by using a feature selection method such as a linear regression model (for example, binary logistic regression) and a nonlinear model (for example, random forest and gradient boost).

The screen configuration processing unit 112 generates display data for displaying, on the display unit 114, a highly effective diagnosis and treatment action or measure calculated according to such a procedure. For example, a calculation result is displayed in the processing result presentation region 502 as shown in FIG. 20. According to FIG. 20, it can be seen that a diagnosis and treatment action or measure that most greatly affects an evaluation index is to take the SGLT2 inhibitor.

In the processing (S304) executed by the disease developing time series information convolution unit 107, with respect to the processing of “extracting an effective diagnosis and treatment action or measure in consideration of time series components in addition to whether the diagnosis and treatment action or measure is executed”, when a data set of explanatory variables is generated, the data set of explanatory variables is generated in consideration of a viewpoint of an early diagnosis and an early treatment while compressing the time series components by weighting and adding instances of events executed in an early stage even when the diagnosis and treatment actions are the same. The processing in step S304 can be implemented by simultaneously introducing processing in steps S302 and S303 when a concept of “early execution” is introduced.

Next, step S307 will be described in detail with reference to FIG. 16.

First, the target disease total cost calculation unit 110 acquires a disease developing date (for example, the disease developing date management table shown in FIG. 10) for each patient from the disease developing event detection unit 104 (S3071).

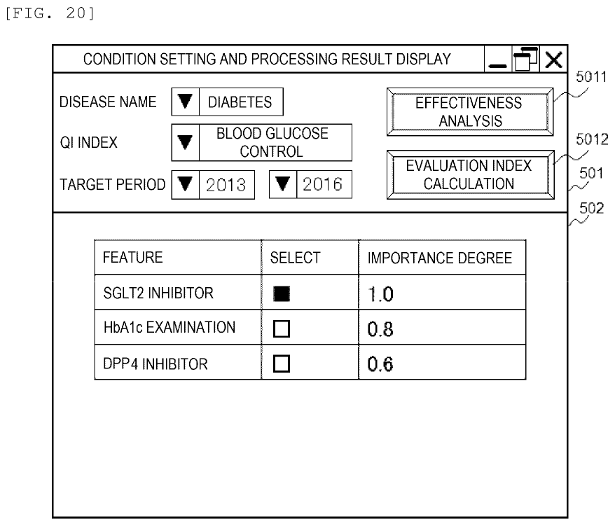

Next, the target disease total cost calculation unit 110 accesses the medical cost database 100 via the external DB cooperation unit 103, and acquires medical costs after a disease developing date for a target patient (S3072). For example, when referring to the disease developing date management table (FIG. 10), since a diabetes developing date of the patient having the patient code P1 is May 1, 2013, medical costs after May 1, 2013 are acquired from the medical cost database 100. That is, medical costs of the patient having the patient code P1 after May 1, 2013 (that is, Jun. 1, 2013, May 1, 2014, and Jun. 1, 2014) are acquired from a medical cost table (FIG. 18).

Finally, the target disease total cost calculation unit 110 calculates total medical costs after the disease developing date for each patient (S3073). The total medical costs calculated at this time may be (1) a sum of all medical costs after the disease developing date, (2) a sum of medical costs of a corresponding disease name after the disease developing date, and (3) a sum of medical costs including that of a disease name (for example, hyperlipidemia which is a complication of diabetes) related to a specified disease after the disease developing date. When medical costs of a related disease are totalized, a disease name related to the corresponding disease may be acquired by referring to a related disease name table shown in FIG. 17. Medical costs for treating a complication or the like can be totalized by totalizing the medical costs of a related disease name.

As shown in FIG. 17, a main disease name and a related disease name are recorded in association with each other in the related disease name table. That is, a patient who has developed a main disease may develop a related disease. For example, a diabetes patient may develop hypertension, dyslipidemia, nephropathy, or the like. Medical costs are totalized with reference to the related disease name table, so that medical costs for treating a complication or the like can be included and totalized, and the medical costs can be accurately totalized by excluding irrelevant medical costs (for example, a diabetes patient needs to pay for a fracture treatment).

The related disease name table may be provided in the target disease total cost calculation unit 110, or may be acquired from an external database.

As shown in FIG. 18, a medical cost table stored in the medical cost database 100 includes a patient attribute (a patient code or the like), a disease name, an execution date, and a medical cost. The medical cost may be recorded in an amount of money, or may be recorded in any unit that can be converted into an amount of money such as the number of insurance points. The medical cost table may also record costs of a measure (for example, health diagnosis, and healthcare guidance) other than a diagnosis and treatment action.

Next, step S308 will be described in detail with reference to FIG. 19.

First, based on whether the diagnosis and treatment action selected on the condition setting and processing result display screen (FIG. 20) is executed, the medical care economic evaluation calculation unit 111 divides target patients into two groups by referring to the diagnosis and treatment action data (FIG. 7) (S3081). According to the diagnosis and treatment action data shown in FIG. 7, a patient having the patient code P0 who has taken the SGLT2 inhibitor is classified into an execution group. On the other hand, a patient having the patient code P1 who has not taken the SGLT2 inhibitor is classified into a non-execution group. Among diagnosis and treatment action data, a diagnosis and treatment action after a disease developing date may be referred to, or all diagnosis and treatment actions may be referred to.

Next, the medical care economic evaluation calculation unit 111 calculates an average value of total medical costs for each group (S3082). Specifically, the medical care economic evaluation calculation unit 111 acquires medical costs of a specified disease name that are totalized for each patient in step S307 and calculates an average value of total medical costs for a patient in each group. Instead of the average value, other statistical processing (for example, calculating a maximum value, a minimum value, a mode value, and a variance) may be performed depending on applications.

For example, when the SGLT2 inhibitor is selected on the condition setting and processing result display screen (FIG. 20), medical costs are displayed on the condition setting and processing result display screen (FIG. 21) in a manner in which a group of patients who takes the SGLT2 inhibitor and a group of patients who does not take the SGLT2 inhibitor can be compared with each other.

FIG. 20 is a diagram showing an example of a condition setting and processing result display screen displayed by the display unit 114 in step S3081.

Similar to the screen shown in FIG. 5, the condition setting and processing result display screen shown in FIG. 20 includes the condition setting region 501 and the processing result presentation region 502. The condition setting region 501 displays the “effectiveness analysis” button 5011 that is operated to analyze effectiveness of a diagnosis and treatment action, the “evaluation index calculation” button 5012 that is operated to evaluate an economic value of a diagnosis and treatment action and measure, and the pull-down selection input fields for setting an analysis condition. In the example showing in FIG. 20, a condition is set for totalizing medical costs of a diagnosis and treatment action or measure having a high effect of affecting blood glucose control by using data of a diabetes patient from year 2013 to year 2016.

As described above, the processing result presentation region 502 displays a highly effective diagnosis and treatment action or measure. According to FIG. 20, it can be seen that a most highly effective diagnosis and treatment action or measure is to take the SGLT2 inhibitor. A “display” button may be provided in the processing result presentation region 502. Although processing of analyzing a diagnosis and treatment action selected by an alternative selection in the selection field is started in the example described above, a plurality of diagnosis and treatment actions can be selected by providing the “display” button. Therefore, when there is a significant difference in diagnosis and treatments due to a combination of a plurality of diagnosis and treatment actions, an effect of the diagnosis and treatment actions (that is, medical costs) can be accurately analyzed. Here, the SGLT2 inhibitor can be selected because a diagnosis and treatment action affecting the quality was automatically selected in S306. In the medical care field, it is unacceptable to evaluate everything by an economic evaluation alone, and it is strongly required to consider medical care quality at the same time. Therefore, an economic evaluation of a diagnosis and treatment action or measure that affects medical care quality can be performed by combining with S306.

FIG. 21 is a diagram showing an example of a condition setting and processing result display screen displayed by the display unit 114 after the processing in step S308 is ended.

Similar to the screen shown in FIG. 20, a condition setting and processing result display screen shown in FIG. 21 includes the condition setting region 501 and the processing result presentation region 502. The screen configuration processing unit 112 generates display data for displaying medical costs in the display unit 114 in a manner in which the execution group and the non-execution group can be compared with each other, and displays the display data in the processing result presentation region 502.

Hereinafter, modifications of the embodiment of the invention will be described.

### <First Modification>

Although one diagnosis and treatment action selected by the user is economically evaluated in the embodiment described above, a plurality of diagnosis and treatment actions are economically evaluated in a first modification.

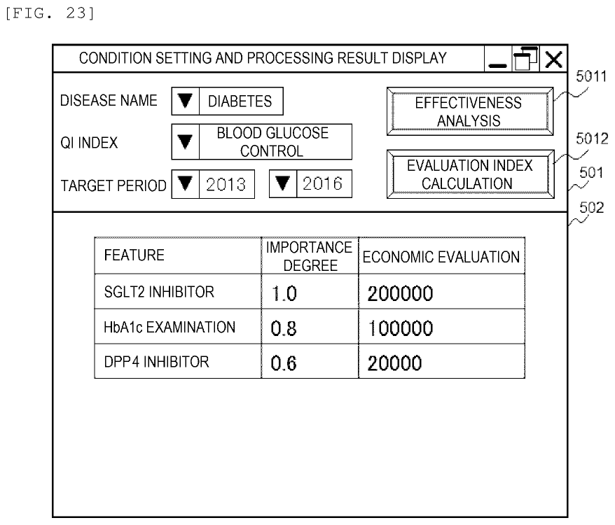

FIG. 22 is a flowchart showing step S308 in detail according to the first modification.

First, the medical care economic evaluation calculation unit 111 selects one diagnosis and treatment action i (S3083). The diagnosis and treatment actions may be selected from the disease developing knowledge data (FIG. 9) or the diagnosis and treatment action data (FIG. 7) in a manner in which the diagnosis and treatment actions i for disease names to be analyzed are not repeated. A table obtained by extracting in advance diagnosis and treatment actions for the disease names to be analyzed from the disease developing knowledge data (FIG. 9) or the diagnosis and treatment action data (FIG. 7) may be created. The diagnosis and treatment actions i may be selected one by one from the created table in step S3083.

Next, based on whether the diagnosis and treatment actions i selected in step S3083 are executed, the medical care economic evaluation calculation unit 111 divides target patients into two groups by referring to the diagnosis and treatment action data (FIG. 7) (S3084). Among diagnosis and treatment action data, a diagnosis and treatment action after a disease developing date may be referred to, or all diagnosis and treatment actions may be referred to.

Then, the medical care economic evaluation calculation unit 111 calculates an average value of total medical costs for each group (S3085). Specifically, the medical care economic evaluation calculation unit 111 acquires medical costs of a specified disease name that are totalized for each patient in step S307 and calculates an average value of total medical costs for a patient in each group. Instead of the average value, other statistical processing (for example, calculating a maximum value, a minimum value, a mode value, and a variance) may be performed depending on applications.

Next, if a parameter i for controlling the diagnosis and treatment actions is smaller than a total number N of the diagnosis and treatment actions, the medical care economic evaluation calculation unit 111 returns the processing to step S3084 and executes the processing for a subsequent diagnosis and treatment action. On the other hand, if the parameter i is equal to or larger than the total number N of the diagnosis and treatment actions, analyses for all the diagnosis and treatment actions are completed and the processing is ended (S3086).

In addition to a loop of the parameter i for the diagnosis and treatment actions, a loop for disease names may be provided, and an average value of total medical costs of a plurality of disease names may be calculated. In this case, an average value of total medical costs of all disease names may be calculated, or an average value of total medical costs of two or more selected disease names may be calculated.

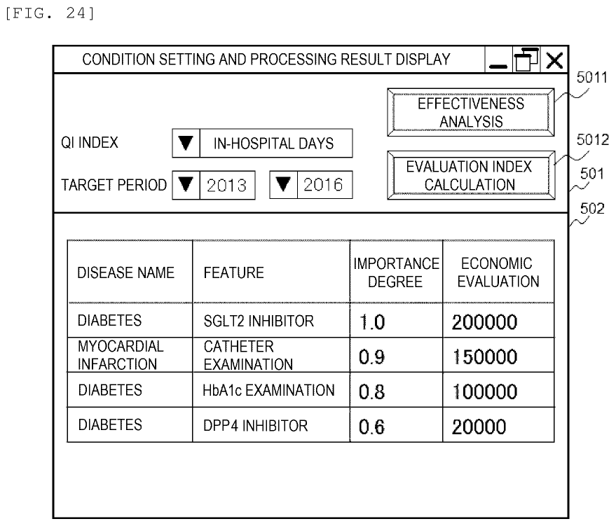

FIG. 23 is a diagram showing an example of a condition setting and processing result display screen displayed by the display unit 114 after the processing in step S308 is ended according to the first modification.

Similar to the screen shown in FIG. 5, the condition setting and processing result display screen shown in FIG. 23 includes the condition setting region 501, the processing result presentation region 502, and the time setting region 503. The condition setting region 501 displays the “effectiveness analysis” button 5011 that is operated to analyze effectiveness of a diagnosis and treatment action, the “evaluation index calculation” button 5012 that is operated to evaluate an economic value of a diagnosis and treatment action or measure, and the pull-down selection input fields for setting an analysis condition. In the example showing in FIG. 23, a condition is set for totalizing medical costs of a diagnosis and treatment action having a high effect of affecting blood glucose control by using data of a diabetes patient from year 2013 to year 2016.

The processing result presentation region 502 displays an economic evaluation of a highly effective diagnosis and treatment action or measure. According to FIG. 23, it can be seen that a diagnosis and treatment action or measure that is highly effective (that is, an importance degree is high in FIG. 23) as a diagnosis and treatment action is to take the SGLT2 inhibitor and the diagnosis and treatment action or measure of taking the SGLT2 inhibitor has the highest economic effect. An economic evaluation may be displayed in an amount of money, or may be displayed in any unit that can be converted into an amount of money such as the number of insurance points.

FIG. 24 is a diagram showing another example of a condition setting and processing result display screen displayed by the display unit 114 after the processing in step S308 is ended according to the first modification. The condition setting and processing result display screen shown in FIG. 24 is displayed by calculating an average value of total medical costs of diagnosis and treatment actions for all disease names in processing shown in FIG. 22.

Similar to the screen shown in FIG. 23, the condition setting and processing result display screen shown in FIG. 24 includes the condition setting region 501, the processing result presentation region 502, and the time setting region 503. The condition setting region 501 displays the “effectiveness analysis” button 5011 that is operated to analyze effectiveness of a diagnosis and treatment action, the “evaluation index calculation” button 5012 that is operated to evaluate an economic value of a diagnosis and treatment action or measure, and the pull-down selection input fields for setting an analysis condition. In the example showing in FIG. 24, a condition is set for totalizing medical costs of a diagnosis and treatment action having a high effect of affecting in-hospital days by using data from year 2013 to year 2016.

The processing result presentation region 502 displays an economic evaluation of a highly effective diagnosis and treatment action or measure. According to FIG. 24, it can be seen that as a diagnosis and treatment action or measure that affects in-hospital days, a diagnosis and treatment action or measure that has a high effect (that is, an importance degree is high in FIG. 24) is to take the SGLT2 inhibitor for treating diabetes and the diagnosis and treatment action or measure of taking the SGLT2 inhibitor also has the highest economic effect. An economic evaluation may be displayed in an amount of money, or may be displayed in any unit that can be converted into an amount of money such as the number of insurance points.

Although medical costs of all disease names are totalized on the screen shown in FIG. 24, an “add” button may be provided on the screen shown in FIG. 23 and a plurality of disease names may be selected. Medical costs of the selected disease names may be totalized to display economic evaluations. Further, only medical costs of related disease names may be totalized to display economic evaluations by using the related disease name table shown in FIG. 17.

Economic evaluations of a plurality of disease names may be displayed in one table as shown in FIG. 24. Alternatively, an economic evaluation of each disease name may be displayed in a table.

As described above, all economic evaluations of a plurality of diagnosis and treatment actions can be viewed according to the first modification, and a diagnosis and treatment action having a high economic effect can be known.

### <Second Modification>

Although patients are divided into two groups based on whether a specified diagnosis and treatment action is executed and medical costs of the two groups are compared with each other in the embodiment described above, patients are divided into two groups according to an execution period of a specified diagnosis and treatment action, and medical costs of the two groups are compared with each other in a second modification.

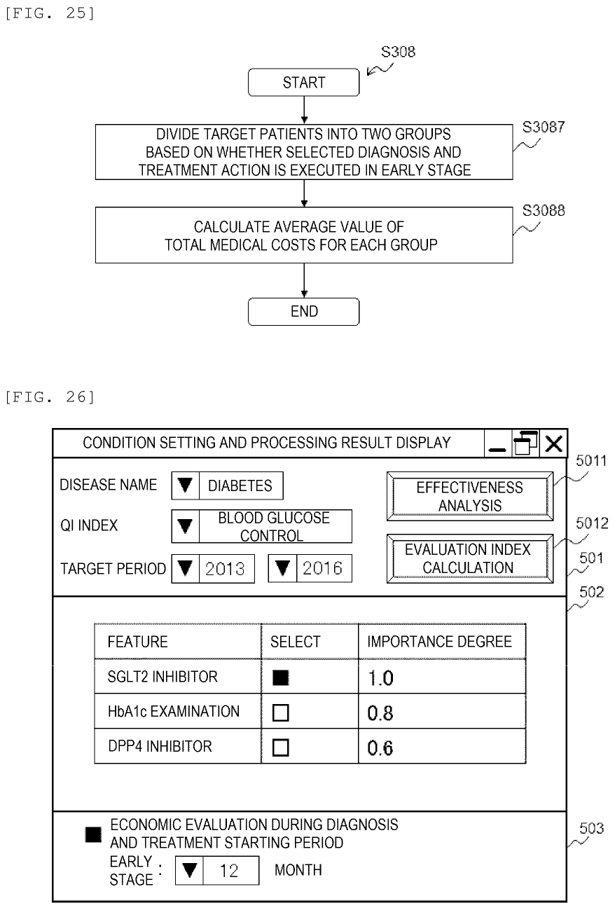

FIG. 25 is a flowchart showing step S308 in detail according to the first modification.

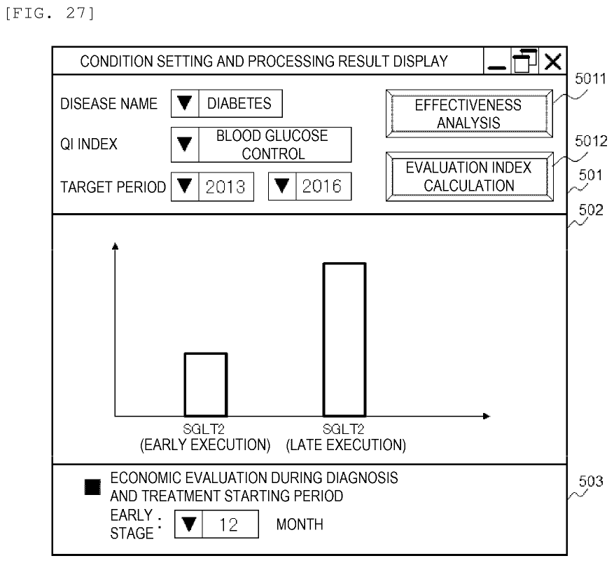

First, based on whether the diagnosis and treatment action selected on the condition setting and processing result display screen (FIG. 20) is executed in an early stage, the medical care economic evaluation calculation unit 111 divides target patients into two groups (including an early execution group and a late execution group) by referring to the diagnosis and treatment action data (FIG. 7) and the disease developing date management table (FIG. 10) (S3087). According to the diagnosis and treatment action data shown in FIG. 7, the patient having the patient code P0 developed diabetes on May 1, 2013 and took the SGLT2 inhibitor on Jun. 1, 2013. Here, an early stage determination criterion is set to 12 months in a condition setting and processing result display screen (FIG. 26). Since 1 month is taken from disease developing to administration of the SGLT2 inhibitor, the patient having the patient code P0 is classified into the early execution group. On the other hand, since the patient having the patient code P1 did not take the SGLT2 inhibitor, the patient is not included in the early execution group or the late execution group. Among diagnosis and treatment action data, a diagnosis and treatment action after a disease developing date may be referred to, or all diagnosis and treatment actions may be referred to.

Next, the medical care economic evaluation calculation unit 111 calculates an average value of total medical costs for each group (S3088). Specifically, the medical care economic evaluation calculation unit 111 acquires medical costs of a specified disease name that are totalized for each patient in step S307 and calculates an average value of total medical costs for a patient in each group. Instead of the average value, other statistical processing (for example, calculating a maximum value, a minimum value, a mode value, and a variance) may be performed depending on applications.

For example, when the SGLT2 inhibitor is selected and an early stage determination criterion is set to 12 months on the condition setting and processing result display screen (FIG. 26), medical costs are displayed on a condition setting and processing result display screen (FIG. 27) in a manner in which medical costs of a group of patients who takes the SGLT2 inhibitor within 12 months after disease developing and medical costs of a group of patients who does not take the SGLT2 inhibitor can be compared with each other.

FIG. 26 is a diagram showing an example of a condition setting and processing result display screen displayed by the display unit 114 in step S3081.

Similar to the screen shown in FIG. 5, the condition setting and processing result display screen shown in FIG. 26 includes the condition setting region 501, the processing result presentation region 502, and the time setting region 503. The condition setting region 501 displays the “effectiveness analysis” button 5011 that is operated to analyze effectiveness of a diagnosis and treatment action, the “evaluation index calculation” button 5012 that is operated to evaluate an economic value of a diagnosis and treatment action or measure, and the pull-down selection input fields for setting an analysis condition. In the example showing in FIG. 26, a condition is set for totalizing medical costs of a diagnosis and treatment action having a high effect of affecting blood glucose control by using data of a diabetes patient from year 2013 to year 2016.

As described above, the processing result presentation region 502 displays a highly effective diagnosis and treatment action or measure. According to FIG. 26, it can be seen that a most highly effective diagnosis and treatment action or measure is to take the SGLT2 inhibitor. A “display” button may be provided in the processing result presentation region 502. Although processing of analyzing a diagnosis and treatment action selected by an alternative selection in the selection field is started in the example described above, a plurality of diagnosis and treatment actions can be selected by providing the “display” button. Therefore, when there is a significant difference in diagnosis and treatments due to a combination of a plurality of diagnosis and treatment actions, an effect of the diagnosis and treatment actions (that is, medical costs) can be accurately analyzed.

An early stage determination criterion (a period from disease developing to when a diagnosis and treatment action is taken) for dividing patients into two groups is set in the time setting region 503. In the example shown in FIG. 26, a condition for determining “an early stage” when a diagnosis and treatment action is executed within 12 months after disease developing is set.

FIG. 27 is a diagram showing an example of a condition setting and processing result display screen displayed by the display unit 114 after the processing in step S308 is ended.

Similar to the screen shown in FIG. 26, the condition setting and processing result display screen shown in FIG. 27 includes the condition setting region 501, the processing result presentation region 502, and the time setting region 503. The screen configuration processing unit 112 generates display data for displaying medical costs on the display unit 114 in a manner in which the early execution group and the late execution group can be compared with each other, and displays the display data in the processing result presentation region 502.

As described above, in the second modification, an economic effect of executing a diagnosis and treatment action in an early stage can be known. A treatment period when the economic effect occurs can be known by setting a period as a parameter and analyzing the parameter.

As described above, according to the embodiment of the invention, since the analysis system includes the input unit 113 configured to receive an analysis condition (such as a period, a disease name, an index); the disease developing event detection unit 104 configured to extract a disease developing event; and the target disease total cost calculation unit 110 configured to calculate costs of a diagnosis and treatment action related to a disease name to be analyzed that are generated after a period of the disease developing event extracted by the disease developing event detection unit 104, the economic value of the clinically highly effective diagnosis and treatment action is not evaluated by unit price of the diagnosis and treatment action itself, but can be presented by the economic effect of the diagnosis and treatment action, and materials that contribute to planning of efficiency improvement of medical costs can be presented. In particular, total medical costs accumulated from the disease developing can be calculated accurately.

Since the target disease total cost calculation unit 110 calculates the costs of the diagnosis and treatment action related to the disease name the same as the disease name to be analyzed that are generated after the period of the disease developing event, the target disease total cost calculation unit 110 can totalize the medical costs not related to the disease to be analyzed, and can present an accurate economic effect for each diagnosis and treatment action.

In addition, since the target disease total cost calculation unit 110 specifies a diagnosis and treatment action of a disease name related to the disease name to be analyzed by referring to related disease name table in which a related disease name is stored, and calculates costs of the diagnosis and treatment action of the disease name to be analyzed and the specified diagnosis and treatment action that are generated after the period of the disease developing event, the target disease total cost calculation unit 110 can totalize medical costs of a (clinically related) disease that may occur due to the disease developing of the disease while excluding unrelated medical costs, and can present an accurate economic effect for each diagnosis and treatment action.

Since the medical care economic evaluation calculation unit 111 totalizes medical costs of the disease name to be analyzed by dividing patients into an execution group of a specified diagnosis and treatment action and a non-execution group of the specified diagnosis and treatment action, an economic effect of each diagnosis and treatment action can be presented in an easy-to-understand manner.

Since the medical care economic evaluation calculation unit 111 totalizes the medical costs of the disease name to be analyzed for each of a plurality of diagnosis and treatment actions by dividing the patients into the execution group of a corresponding diagnosis and treatment action and the non-execution group of the diagnosis and treatment action, all economic evaluations of the plurality of diagnosis and treatment actions can be viewed, and a diagnosis and treatment action having a high economic effect can be known.

Since the medical care economic evaluation calculation unit 111 totalizes medical costs of all disease names to be analyzed or a selected disease name to be analyzed for each of a plurality of diagnosis and treatment actions by dividing the patients into the execution group of the corresponding diagnosis and treatment action and the non-execution group of the diagnosis and treatment action, and the screen configuration processing unit 112 generates display data for displaying economic evaluations of the plurality of diagnosis and treatment actions in a descending order of a difference between the totalized medical costs in the execution group and the totalized medical costs in the non-execution group, all economic evaluations of the plurality of diagnosis and treatment actions can be viewed, and a diagnosis and treatment action having a high economic effect can be known.

In addition, since the medical care economic evaluation calculation unit 111 totalizes medical costs of the disease name to be analyzed by dividing patients into a group in which a specified diagnosis and treatment action is executed within a predetermined period from the disease developing event and a group in which the specified diagnosis and treatment action is executed after the predetermined period, an economic effect of executing a diagnosis and treatment action in an early stage can be known. A treatment period when the economic effect occurs can be known by setting a period as a parameter and analyzing the parameter.

In addition, since the analysis system includes the disease developing and diagnosis and treatment action relationship extraction unit 106 configured to calculate a time series relationship between a period of the disease developing event extracted by the event detection unit and an execution period of the diagnosis and treatment action and measure; a feature generation unit (the disease developing time series information convolution unit 107) configured to generate a feature amount of the diagnosis and treatment action and measure in consideration of the time series relationship based on the time series relationship calculated by the disease developing and diagnosis and treatment action relationship extraction unit 106 and an execution amount of the diagnosis and treatment action and measure; the evaluation index calculation unit 108 configured to calculate an index value indicating medical care quality based on clinical data including a history of the diagnosis and treatment action and measure and an examination result of a patient; and the diagnosis and treatment effect extraction unit 109 configured to extract a diagnosis and treatment action and measure having a good index value by setting the feature amount of the diagnosis and treatment action and measure extracted by the disease developing time series information convolution unit 107 as an explanatory variable and the index value calculated by the evaluation index calculation unit 108 as an objective variable, even when the disease developing event is lost in the database created by one engine, the disease developing event can be accurately estimated and relative time between the disease developing event and an execution date of the diagnosis and treatment action or measure can be calculated. Further, by reflecting the time series components in the explanatory variables without increasing the number of explanatory variables, the over-learning can be reduced, and a model describing a diagnosis and treatment action effect capable of reducing calculation time can be created.

The invention is not limited to the embodiment described above, and includes various modifications and equivalent configurations within the scope of the appended claims. For example, the embodiment described above has been described in detail for easy understanding of the invention, and the invention is not necessarily limited to those having all the configurations described above. A part of configurations of one embodiment can be replaced with another configuration. The configuration of one embodiment can also be added to the configuration of another embodiment. A part of the configurations of the embodiment may be added to, deleted from, and replaced with another configuration.

The configurations, functions, processing units, processing methods, and the like described above may be partially or entirely implemented by hardware such as through a design using an integrated circuit, or may be implemented by software by a processor interpreting and executing programs for realizing respective functions.

Information such as a program, a table, and a file for realizing each function can be stored in a storage device such as a memory, a hard disk, and a solid state drive (SSD), or a recording medium such as an IC card, an SD card, and a DVD.

Control lines and information lines indicate those that are considered necessary for description, and not all the control lines and the information lines are necessarily shown in a product. In practice, it may be considered that almost all the configurations are connected to one another.

### INDUSTRIAL APPLICABILITY

The invention relates to a technique of a hospital information system in a medical field, and is particularly useful as a technique for supporting an effect analysis of a diagnosis and treatment action or measure.

1. An analysis system configured to analyze an effect of a diagnosis and treatment action and measure and configured to be implemented by a computer including an arithmetic device configured to execute predetermined processing and a storage device connected to the arithmetic device, the analysis system comprising:
an input unit configured to receive an analysis condition;
an event detection unit configured to extract a disease developing event; and
a cost calculation unit configured to calculate a cost of a diagnosis and treatment action related to a disease name to be analyzed that is generated after a period of the disease developing event extracted by the event detection unit.

2. The analysis system according to claim 1, wherein
the cost calculation unit calculates the cost of the diagnosis and treatment action related to a disease name the same as the disease name to be analyzed that is generated after the period of the disease developing event.

3. The analysis system according to claim 1, wherein
the cost calculation unit specifies a diagnosis and treatment action of a disease name related to the disease name to be analyzed by referring to related disease name information in which a related disease name is stored, and
calculates costs of the diagnosis and treatment action of the disease name to be analyzed and the specified diagnosis and treatment action that are generated after the period of the disease developing event.

4. The analysis system according to claim 1, further comprising:
an economic evaluation calculation unit configured to totalize medical costs of the disease name to be analyzed by dividing patients into an execution group of a specified diagnosis and treatment action and a non-execution group of the specified diagnosis and treatment action.

5. The analysis system according to claim 4, wherein
the economic evaluation calculation unit totalizes the medical costs of the disease name to be analyzed for each of a plurality of diagnosis and treatment actions by dividing the patients into the execution group of a corresponding diagnosis and treatment action and the non-execution group of the diagnosis and treatment action.

6. The analysis system according to claim 5, further comprising:
a screen configuration processing unit configured to generate screen display data to be output to a display device, wherein
the economic evaluation calculation unit totalizes medical costs of all disease names to be analyzed or a selected disease name to be analyzed for each of a plurality of diagnosis and treatment actions by dividing the patients into the execution group of the corresponding diagnosis and treatment action and the non-execution group of the diagnosis and treatment action, and
the screen configuration processing unit generates display data for displaying economic evaluations of the plurality of diagnosis and treatment actions in a descending order of a difference between the totalized medical costs in the execution group and the totalized medical costs in the non-execution group.

7. The analysis system according to claim 1, further comprising:
an economic evaluation calculation unit configured to totalize medical costs of the disease name to be analyzed by dividing patients into a group in which a specified diagnosis and treatment action is executed within a predetermined period from the disease developing event and a group in which the specified diagnosis and treatment action is executed after the predetermined period.

8. The analysis system according to claim 1, further comprising:
a relationship extraction unit configured to calculate a time series relationship between a period of the disease developing event extracted by the event detection unit and an execution period of the diagnosis and treatment action and measure;
a feature generation unit configured to generate a feature amount of the diagnosis and treatment action and measure in consideration of the time series relationship based on the time series relationship calculated by the relationship extraction unit and an execution amount of the diagnosis and treatment action and measure;
an index calculation unit configured to calculate an index value indicating medical care quality based on clinical data including a history of the diagnosis and treatment action and measure and an examination result of a patient; and
an effect extraction unit configured to extract a diagnosis and treatment action and measure having a good index value by setting the feature amount of the diagnosis and treatment action and measure extracted by the feature generation unit as an explanatory variable and the index value calculated by the index calculation unit as an objective variable.

9. An analysis method for analyzing an effect of a diagnosis and treatment action and measure by a computer including an arithmetic device configured to execute predetermined processing and a storage device connected to the arithmetic device,
the analysis method comprising:
an input step of receiving, by the arithmetic device, an analysis condition;
an event detection step of extracting, by the arithmetic device, a disease developing event; and
a cost calculation step of calculating, by the arithmetic device, a cost of a diagnosis and treatment action related to a disease name to be analyzed that is generated after a period of the disease developing event extracted in the event detection step.

10. The analysis method according to claim 9, wherein
in the cost calculation step, the arithmetic device calculates a cost of a diagnosis and treatment action related to a disease name the same as the disease name to be analyzed that is generated after the period of the disease developing event.

11. The analysis method according to claim 9, wherein
in the cost calculation step,
the arithmetic device specifies a diagnosis and treatment action of a disease name related to the disease name to be analyzed by referring to related disease name information in which a related disease name is stored, and
the arithmetic device calculates costs of the diagnosis and treatment action of the disease name to be analyzed and the specified diagnosis and treatment action that are generated after the period of the disease developing event.

12. The analysis method according to claim 9, further comprising:
an economic evaluation calculation step of totalizing, by the arithmetic device, medical costs of the disease name to be analyzed by dividing patients into an execution group of a specified diagnosis and treatment action and a non-execution group of the specified diagnosis and treatment action.

13. The analysis method according to claim 12, wherein
in the economic evaluation calculation step, the arithmetic device totalizes medical costs of the disease name to be analyzed for each of a plurality of diagnosis and treatment actions by dividing patients into an execution group and a non-execution group.

14. The analysis method according to claim 13, further comprising:
a screen configuration processing step of generating, by the arithmetic device, screen display data to be output to a display device, wherein
in the economic evaluation calculation step, the arithmetic device totalizes medical costs of all disease names to be analyzed or a selected disease name to be analyzed for each of a plurality of diagnosis and treatment actions by dividing the patients into the execution group of a corresponding diagnosis and treatment action and the non-execution group of the diagnosis and treatment action, and
in the screen configuration processing step, the arithmetic device generates display data for displaying economic evaluations of the plurality of diagnosis and treatment actions in a descending order of a difference between totalized medical costs in the execution group and totalized medical costs in the non-execution group.

15. The analysis method according to claim 9, further comprising:
an economic evaluation calculation step of totalizing, by the arithmetic device, medical costs of the disease name to be analyzed by dividing patients into a group in which a specified diagnosis and treatment action is executed within a predetermined period from the disease developing event and a group in which the specified diagnosis and treatment action is executed after the predetermined period.
